(19) United States
(12) Patent Application Publication
(10) Pub. No.: US 2019/0126009 A1
(43) Pub. Date: May 2, 2019

(54) STEERING ASSEMBLY FOR INTRAVASCULAR CATHETER SYSTEM

(71) Applicant: CRYTERION MEDICAL, INC., Carlsbad, CA (US)

(72) Inventor: Eric A. Schultheis, San Clemente, CA (US)

(21) Appl. No.: 16/115,346

(22) Filed: Aug. 28, 2018

Related U.S. Application Data
(60) Provisional application No. 62/580,097, filed on Nov. 1, 2017.

(51) Int. Cl. A61M 25/01; A61M 25/00; A61M 25/10

(52) U.S. Cl. CPC A61M 25/0147; A61M 25/0021; A61M 25/1018; A61M 25/0074

(57) ABSTRACT

A steering assembly for an intravascular catheter system includes a steering mechanism, a steering anchor and a first pull wire. The intravascular catheter system includes an inflatable balloon and a guidewire lumen that extends through the inflatable balloon. The guidewire lumen has a distal region. The steering mechanism is positioned away from the inflatable balloon. The steering anchor can be secured to the distal region of the guidewire lumen. The first pull wire is secured to the steering anchor and coupled to the steering mechanism so that actuation of the steering mechanism articulates the distal region of the guidewire lumen. The steering assembly can further include a second pull wire that is also secured to the steering anchor and coupled to the steering mechanism.

### RELATED APPLICATION

This application claims priority on U.S. Provisional Application Ser. No. 62/580,097 filed on Nov. 1, 2017 and entitled “BALLOON CATHETER STEERING ASSEMBLY FOR A CRYOGENIC BALLOON CATHETER SYSTEM”.

As far as permitted, the contents of U.S. Provisional Application Ser. No. 62/580,097 are incorporated in their entirety herein by reference.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning a portion of an energy delivery catheter adjacent to diseased or targeted tissue in the heart. The energy delivery component of the system is typically at or near a most distal (farthest from the operator) portion of the catheter, and often at a tip of the device. Various forms of energy are used to ablate diseased heart tissue. These can include radio frequency (RF), ultrasound and laser energy, to name a few. One form of energy that is used to ablate diseased heart tissue includes cryogenics (also referred to herein as “cryoablation”). During a cryoablation procedure, with the aid of a guidewire, the distal tip of the catheter is positioned adjacent to diseased or targeted tissue, at which time the cryogenic energy can be delivered to create tissue necrosis, rendering the ablated tissue incapable of conducting electrical signals.

Atrial fibrillation is one of the most common arrhythmias treated using cryoablation. In the earliest stages of the disease, paroxysmal atrial fibrillation, the treatment strategy involves isolating the pulmonary veins from the left atrial chamber, a procedure that removes unusual electrical conductivity in the pulmonary vein. Recently, the use of techniques known as “balloon cryotherapy” catheter procedures to treat atrial fibrillation have increased. In part, this stems from ease of use, shorter procedure times and improved patient outcomes. During the balloon cryotherapy procedure, a refrigerant or cryogenic fluid (such as nitrous oxide, or any other suitable fluid) is delivered under pressure to an interior of one or more inflatable balloons which are positioned adjacent to or against the targeted cardiac tissue. Using this method, the extremely frigid cryogenic fluid causes necrosis of the targeted cardiac tissue, thereby rendering the ablated tissue incapable of conducting unwanted electrical signals.

During cryoablation procedures, the distal end of the catheter is designed to reach tissue within the patient's heart. In order to reach various locations within the heart, the procedure requires that the catheter be carefully steered or navigated through the patient's body, particularly the patient's vascular path. Navigation of the catheter is generally performed with the use of pull wire(s) that typically extend from within a handle assembly and run distally through the wall of a catheter sheath and/or catheter shaft. Specifically, manipulating the pull wire(s) causes a distal end of the catheter to articulate, allowing the catheter to be steered, navigated and/or ultimately positioned advantageously in a region of interest for the cryoablation procedure.

### SUMMARY

The present invention is directed toward a steering assembly for an intravascular catheter system. In certain embodiments, the intravascular system can include an inflatable balloon and a guidewire lumen that extends through the inflatable balloon. The guidewire lumen can also have a distal region. In various embodiments, the steering assembly can include a steering mechanism, a steering anchor and a first pull wire. The steering mechanism is positioned away from the inflatable balloon. The steering anchor is secured to the distal region of the guidewire lumen. In certain embodiments, the first pull wire can be secured to the steering anchor and coupled to the steering mechanism so that actuation of the steering mechanism articulates the distal region of the guidewire lumen.

In certain embodiments, the intravascular catheter system can include a handle assembly. In such embodiments, the steering mechanism can be positioned within the handle assembly.

In some embodiments, the distal region of the guidewire lumen can include a distal tip. In one embodiment, the steering anchor can be secured to the distal tip.

In various embodiments, the steering anchor can be secured to an interior of the guidewire lumen. Alternatively, the steering anchor can secured to an exterior of the guidewire lumen. Additionally, in certain embodiments, the first pull wire can be positioned within the interior of the guidewire lumen.

In certain embodiments, the steering mechanism can move the first pull wire to articulate the distal region of the guidewire lumen.

In some embodiments, the steering assembly can further include a second pull wire that is secured to the steering anchor and coupled to the steering mechanism. In such embodiments, the steering anchor can be secured to the distal tip. Alternatively, the steering anchor can be secured to the interior of the guidewire lumen. Still alternatively, the steering anchor can be secured to the exterior of the guidewire lumen. Additionally, the first pull wire and the second pull wire can be positioned within the interior of the guidewire lumen. In certain embodiments, the steering mechanism can move the first pull wire and the second pull wire to articulate the distal region of the guidewire lumen.

The present invention is further directed toward a steering assembly for an intravascular catheter system. In certain embodiments, the intravascular system can include an inflatable balloon and a guidewire lumen that extends through the inflatable balloon. The guidewire lumen can also have a distal region. In certain embodiments, the steering assembly can include a steering mechanism, a first pull wire and a second pull wire. Both the first pull wire and the second pull wire can be coupled to the steering mechanism and connected to the distal region of the guidewire lumen such that actuation of the steering mechanism articulates the distal region of the guidewire lumen.

In some embodiments, the distal region of the guidewire lumen can include a distal tip. In such embodiments, the first pull wire and the second pull wire can be connected to the distal tip.

In other embodiments, the first pull wire and the second pull wire can positioned within an interior of the guidewire lumen.

In addition, in various embodiments, the steering mechanism can move the first pull wire and the second pull wire to articulate the distal region of the guidewire lumen.

In various embodiments, the steering assembly can further include a steering anchor positioned within the distal region of the guidewire lumen. In such embodiments, the steering anchor can be secured either the interior of the guidewire lumen or an exterior of the guidewire lumen.

In certain applications, the present invention is further directed toward a steering assembly for an intravascular catheter system. In certain embodiments, the intravascular system can include a handle assembly, an inflatable balloon and a guidewire lumen that extends through the inflatable balloon. The guidewire lumen has a distal region. In various embodiments, the steering assembly can include a steering mechanism that is positioned within the handle assembly, a steering anchor, a first pull wire and a second pull wire. The steering anchor can be positioned within the distal region of the guidewire lumen and secured to an interior of the guidewire lumen. Both the first pull wire and the second pull wire can be secured to the steering anchor and coupled to the steering mechanism such that actuation of the steering mechanism articulates the distal region of the guidewire lumen.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of a patient and one embodiment of an intravascular catheter system having features of the present invention;

FIG. 2 is a simplified side view of an embodiment of a portion of the intravascular catheter system, including an embodiment of a catheter steering assembly; and

FIG. 3 is a simplified side view of another embodiment of a portion of the intravascular catheter system, including another embodiment of the catheter steering assembly.

### DESCRIPTION

Embodiments of the present invention are described herein in the context of a catheter steering assembly (also sometimes referred to herein as a “steering assembly”) for an intravascular catheter system. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound, pulsed DC electric fields and laser energy, as non-exclusive examples. The present invention is intended to be effective with any or all of these and other forms of energy.

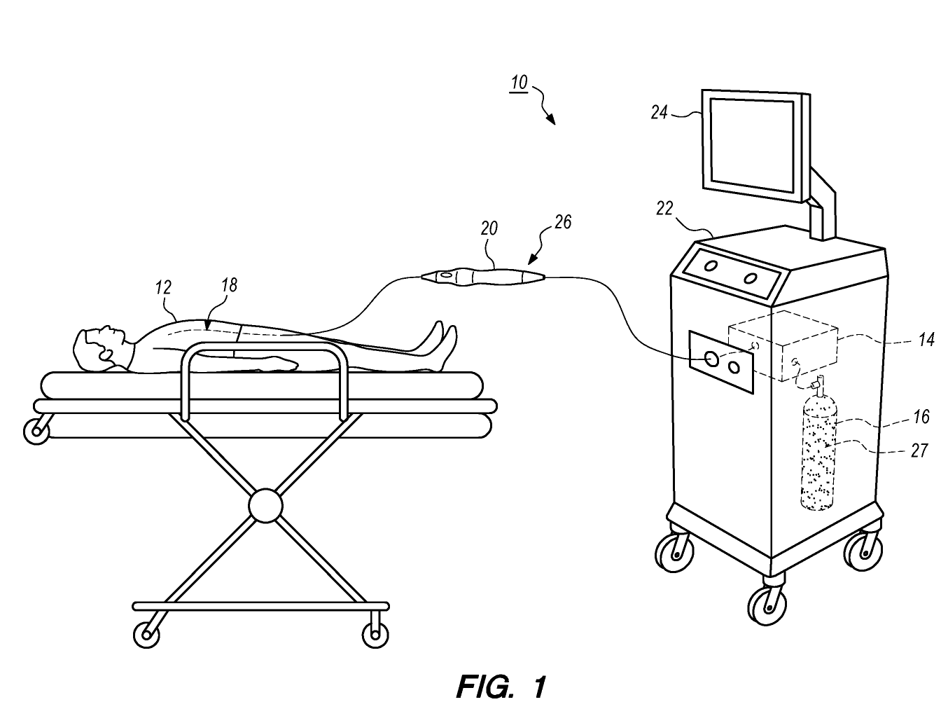

FIG. 1 is a schematic view of one embodiment of an intravascular catheter system 10 (also sometimes referred to as a “catheter system”) for use with a patient 12, which can be a human being or an animal. Although the catheter system 10 is specifically described herein with respect to the intravascular catheter system, it is understood and appreciated that other types of catheter systems and/or ablation systems can equally benefit by the teachings provided herein. For example, in certain non-exclusive alternative embodiments, the present invention can be equally applicable for use with any suitable types of ablation systems and/or any suitable types of catheter systems. Thus, the specific reference herein to use as part of the intravascular catheter system is not intended to be limiting in any manner.

The design of the catheter system 10 can be varied. In certain embodiments, such as the embodiment illustrated in FIG. 1, the catheter system 10 can include one or more of a control system 14, a fluid source 16 (e.g., one or more fluid containers), a balloon catheter 18, a handle assembly 20, a control console 22, a graphical display 24 (also sometimes referred to as a graphical user interface or “GUI”) and a steering assembly 26. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional structures than those specifically illustrated and described herein.

In various embodiments, the control system 14 is configured to monitor and control the various processes of a cryoablation procedure. More specifically, the control system 14 can monitor and control release and/or retrieval of a cryogenic fluid 27 to and/or from the balloon catheter 18. The control system 14 can also control various structures that are responsible for maintaining or adjusting a flow rate and/or a pressure of the cryogenic fluid 27 that is released to the balloon catheter 18 during the cryoablation procedure. In such embodiments, the catheter system 10 delivers ablative energy in the form of cryogenic fluid 27 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive electrical signals, data and/or other information (also sometimes referred to as “sensor output”) from various structures within the catheter system 10. In various embodiments, the control system 14 and/or the GUI 24 can be electrically connected and/or coupled. In some embodiments, the control system 14 can receive, monitor, assimilate and/or integrate any sensor output and/or any other data or information received from any structure within the catheter system 10 in order to control the operation of the balloon catheter 18. Still further, or in the alternative, the control system 14 can control positioning of portions of the balloon catheter 18 within a circulatory system (not shown) (also sometimes referred to herein as the “body”) of the patient 12, and/or can control any other suitable functions of the balloon catheter 18.

The fluid source 16 (also sometimes referred to as “fluid container 16”) can include one or more fluid container(s) 16. It is understood that while one fluid container 16 is illustrated in FIG. 1, any suitable number of fluid containers 16 may be used. The fluid container(s) 16 can be of any suitable size, shape and/or design. The fluid container(s) 16 contains the cryogenic fluid 27, which is delivered to the balloon catheter 18 with or without input from the control system 14 during the cryoablation procedure. Once the cryoablation procedure has initiated, the cryogenic fluid 27 can be injected or delivered and the resulting gas, after a phase change, can be retrieved from the balloon catheter 18, and can either be vented or otherwise discarded as exhaust (not shown). More specifically, the cryogenic fluid 27 delivered to and/or removed from the balloon catheter 18 can include a flow rate that varies. Additionally, the type of cryogenic fluid 27 that is used during the cryoablation procedure can vary. In one non-exclusive embodiment, the cryogenic fluid 27 can include liquid nitrous oxide. In another non-exclusive embodiment, the cryogenic fluid 27 can include liquid nitrogen. However, any other suitable cryogenic fluid 27 can be used.

The design of the balloon catheter 18 can be varied to suit the design requirements of the catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a qualified healthcare professional (also referred to herein as an “operator”). As used herein, healthcare professional and/or operator can include a physician, a physician's assistant, a nurse and/or any other suitable person or individual. In certain embodiments, the balloon catheter 18 is positioned within the body of the patient 12 utilizing at least a portion of the sensor output that is received from the balloon catheter 18. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the operator to operate, position and control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid container 16 and the GUI 24. In some embodiments, the handle assembly 20 can integrate and/or include at least a portion of the control system 14 and/or steering assembly 26 within an interior of the handle assembly 20. In one embodiment, an operator can steer and/or navigate the balloon catheter 18 by utilizing the handle assembly 20 and/or the steering assembly 26. It is understood that the handle assembly 20 can include fewer or additional components than those specifically illustrated and described herein.

In the embodiment illustrated in FIG. 1, the control console 22 includes at least a portion of the control system 14, the fluid container 16 and/or the GUI 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the GUI 24.

In various embodiments, the GUI 24 is electrically connected to the control system 14. Additionally, the GUI 24 provides the operator of the catheter system 10 with information that can be used before, during and after the cryoablation procedure. For example, the GUI 24 can provide the operator with information based on the sensor output, and any other relevant information that can be used before, during and after the cryoablation procedure. The specifics of the GUI 24 can vary depending upon the design requirements of the catheter system 10, or the specific needs, specifications and/or desires of the operator.

In one embodiment, the GUI 24 can provide static visual data and/or information to the operator. In addition, or in the alternative, the GUI 24 can provide dynamic visual data and/or information to the operator, such as video data or any other data that changes over time, e.g., during the cryoablation procedure. Further, in various embodiments, the GUI 24 can include one or more colors, different sizes, varying brightness, etc., that may act as alerts to the operator. Additionally, or in the alternative, the GUI 24 can provide audio data or information to the operator.

As an overview, and as provided in greater detail herein, the steering assembly 26 can be configured to articulate a portion of the balloon catheter 18, allowing the balloon catheter 18 to be steered, navigated and/or ultimately positioned within the body of the patient 12 during the cryoablation procedure. As used herein, it is understood that the term “articulate” can include bend, turn, deflect, curve, or any other non-linear movement. In the embodiment illustrated in FIG. 1, at least a portion of the steering assembly 26 is integrated with and/or positioned on or within the handle assembly 20 and/or the balloon catheter 18. The steering assembly 26 can be positioned at any suitable location on or within the handle assembly 20 and/or the balloon catheter 18. Additionally, and/or in the alternative, at least a portion of the steering assembly 26 can be integrated with and/or positioned on or within any other suitable structure of the catheter system 10.

The specific components and operations of the steering assembly 26 will be described in greater detail herein in relation to the embodiments illustrated in the drawings. It is appreciated that the drawings included herewith may not necessarily be drawn to scale. Additionally, it is further appreciated that the drawings may not precisely represent the structures or components of the catheter system 10 and/or steering assembly 26, but are included for purposes of clarity in demonstrating certain features and limitations of the catheter system 10 and/or steering assembly 26.

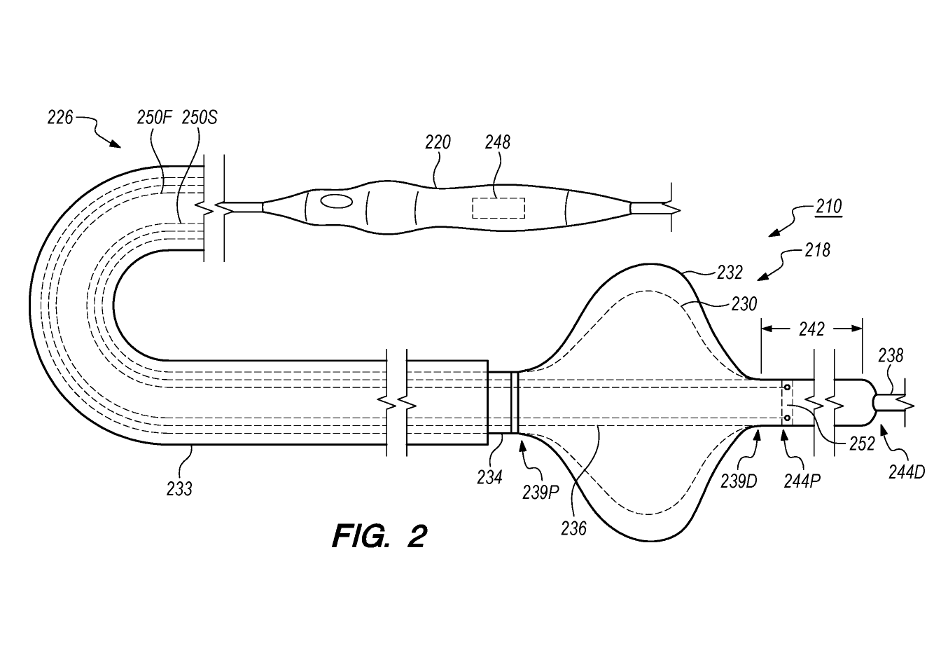

FIG. 2 is a simplified side view of an embodiment of a portion of the catheter system 210, including an embodiment of the steering assembly 226. In the embodiment illustrated in FIG. 2, the catheter system 210 can include one or more of the balloon catheter 218, the handle assembly 220 and the steering assembly 226.

In the embodiment illustrated in FIG. 2, at least a portion of the balloon catheter 218 can be inserted into the body of the patient 12 (illustrated in FIG. 1) during the cryoablation procedure. The design of the balloon catheter 218 can be varied to suit the design requirements of the catheter system 210. In this embodiment, the balloon catheter 218 includes one or more of an inner inflatable balloon 230, an outer inflatable balloon 232, a catheter sheath 233, a catheter shaft 234, a guidewire lumen 236 and a guidewire 238. It is understood that the balloon catheter 218 can include other structures as well that are not shown and/or described relative to FIG. 2.

In the embodiment illustrated in FIG. 2, the outer inflatable balloon 232 substantially encircles the inner inflatable balloon 230. During use, the inner inflatable balloon 230 can be partially or fully inflated so that at least a portion of the inner inflatable balloon 230 expands toward and/or against a portion of the outer inflatable balloon 232 (although a space is shown between the inner inflatable balloon 230 and the outer inflatable balloon 232 in FIG. 2 for clarity). In various embodiments, the inner inflatable balloon 230 and the outer inflatable balloon 232 can be at least partially secured to the catheter shaft 234 and/or the guidewire lumen 236. In FIG. 2, the inflatable balloons 230, 232, can both include a proximal balloon region 239P and a distal balloon region 239D. The proximal balloon region 239P includes the region closer to the handle assembly 220, where at least a portion of one or more inflatable balloons 230, 232, are secured or attached to a portion of the catheter shaft 234. Further, the distal balloon region 239D includes the region further away from the handle assembly 220, where at least a portion of one or more inflatable balloons 230, 232, are secured or attached to the guidewire lumen 236. Alternatively, the proximal balloon region 239P and/or the distal balloon region 239D can be secured or attached to other suitable structures within the catheter system 210. The proximal balloon region 239P and/or the distal balloon region 239D can be secured or attached to the guidewire lumen 236, the catheter shaft 234 and/or any other suitable structures, via any manner or method, such as with an adhesive or heat-bonding, as non-exclusive examples.

The guidewire 238 is inserted into the body of the patient 12, and the guidewire lumen 236 is moved along the guidewire 238 to near an ostium (not shown) of a pulmonary vein (not shown) of the patient 12. More particularly, the guidewire lumen 236 encircles the guidewire 238. During use, the guidewire 238 can at least partially extend through the guidewire lumen 236. Additionally, the guidewire 238 and/or guidewire lumen 236 can be inserted into the body of the patient 12, and the catheter shaft 234 can be moved along the guidewire 238 and/or guidewire lumen 236 to near the ostium of the pulmonary vein of the patient 12. The catheter sheath 233 can also be moved along catheter shaft 234 to near the ostium of the pulmonary vein of the patient 12. In various embodiments, the guidewire 238, guidewire lumen 236, catheter shaft 234 and/or catheter sheath 233 can extend between the handle assembly 220 to at or near the ostium of the pulmonary vein of the patient 12.

The design of the guidewire lumen 236 can vary. In various embodiments, the guidewire lumen 236 extends through the inflatable balloons 230, 232. In some embodiments, the guidewire lumen 236 can include a guidewire lumen distal region 242 (also sometimes referred to herein as a “distal region”). As used herein, the distal region 242 is the portion of the guidewire lumen 236 that is first inserted into the patient 12. Additionally, as referred to herein, the term “distal” can include any location on the guidewire lumen 236 that is away from and/or further away from the handle assembly 220. For example, in this embodiment, the distal region 242 of the guidewire lumen 236 includes the portion of the guidewire lumen 236 that is distal to the distal balloon region 239D.

The distal region 242 of the guidewire lumen 236 can further include a proximal end 244P and a distal tip 244D. As used herein, the proximal end 244P can include the portion of the distal region 242 at or near the distal balloon region 239D and/or any other location nearer to the handle assembly 220 than the distal tip 244D. The distal tip 244D can include the portion of the distal region 242 at or near the end or tip of the guidewire lumen 236, i.e., at or near the location where the guidewire 238 exits the guidewire lumen 236.

The steering assembly 226 can allow the balloon catheter 218 to be articulated in order to steer, navigate and/or advantageously position the balloon catheter 218 during the cryoablation procedure. More specifically, the steering assembly 226 can be configured to articulate at least a portion of the balloon catheter 218 at or near the distal region 242 of the guidewire lumen 236. The design of the steering assembly 226 can vary. In the embodiment illustrated in FIG. 2, the steering assembly 226 can include one or more of a steering mechanism 248, a first pull wire 250F, a second pull wire 250S and a steering anchor 252. It is understood that the steering assembly 226 can include fewer or additional components than those specifically illustrated and described herein.

The steering mechanism 248 is configured to allow the operator to articulate at least a portion of the balloon catheter 218, i.e., the distal region 242 of the guidewire lumen 236, during the cryoablation procedure. The design and/or configuration of the steering mechanism 248 can vary. For example, articulation of at least a portion of the balloon catheter 218 can be realized through actuation of individual and/or collective workings of various components of the steering mechanism 248. As used herein, the term actuate can include to operate, activate, control, maneuver, direct, rotate, push, pull, turn, etc. In certain embodiments, the steering mechanism 248 can include one or more of: steering member(s) (not shown), such as a knob or switch; drive member(s) (not shown), such as slides, screws, racks, etc.; pulley(s) (not shown); and/or gear(s) (not shown); as non-exclusive examples. It is understood that the steering mechanism 248 can include fewer or additional components than those specifically described herein. Additionally, and/or alternatively, the steering mechanism 248 can include any other design and/or configuration that allows the operator to articulate at least a portion of the balloon catheter 218 during the cryoablation procedure. In some embodiments, such as the embodiment illustrated in FIG. 2, the steering mechanism 248 is positioned away from the inflatable balloons 230, 232. Additionally, as shown in FIG. 2, in certain embodiments the steering mechanism 248 can be integrated and/or included as part of the handle assembly 220. In such embodiments, the steering mechanism 248 can be fully and/or partially positioned within the handle assembly 220. In other embodiments, the steering mechanism 248 can be positioned away from the inflatable balloons 230, 232, but separate and/or apart from the handle assembly 220.

In certain embodiments, the first pull wire 250F can extend generally between the steering mechanism 248 and the distal region 242 of the guidewire lumen 236. Furthermore, the second pull wire 250S can also extend generally between the steering mechanism 248 and the distal region 242 of the guidewire lumen 236. The pull wires 250F, 250S, can be coupled, secured or connected to the handle assembly 220 and/or steering mechanism 248, which may allow the pull wires 250F, 250S, to be maneuvered or controlled by the operator to articulate the guidewire lumen 236, to ultimately position the balloon catheter 218 at or near the ostium of the pulmonary vein of the patient 12 during the cryoablation procedure. The pull wires 250F, 250S, can be coupled, secured or connected to the handle assembly 220 and/or the steering mechanism 248 in any suitable manner.

While the embodiment illustrated in FIG. 2 only shows the first and second pull wires 250F, 250S, it is understood that the steering assembly 226 can include a greater or a fewer number of pull wires 250F, 250S. However, for ease in understanding, two pull wires 250F, 250S, are shown and described herein. In the embodiment illustrated in FIG. 2, the pull wires 250F, 250S, extend between the steering mechanism 248 and the proximal end 244P of the distal region 242 of the guidewire lumen 236.

In various embodiments, portions of the pull wires 250F, 250S, can be positioned within an interior of the guidewire lumen 236. As referred to herein, the interior of the guidewire lumen 236 can include a cavity or channel or a wall of the guidewire lumen 236, as non-exclusive examples. In some embodiments, the pull wires 250F, 250S, can be embedded within the wall. Additionally, in some embodiments, a dedicated lumen for the pull wires 250F, 250S, may also be positioned within the interior of the guidewire lumen 236. In various embodiments, the pull wires 250F, 250S, can be coupled, secured and/or connected to the interior of the guidewire lumen 236. For example, in one embodiment, the pull wires 250F, 250S, can be coupled, secured and/or connected to an inner surface or wall of the guidewire lumen 236. The pull wires 250F, 250S, can be coupled, secured and/or connected to the interior of the guidewire lumen 236 in any suitable manner, i.e., weld or solder joint, adhesive, bonding material, etc., as non-exclusive examples. Alternatively, the pull wires 250F, 250S, may be coupled, secured and/or connected to the interior of the guidewire lumen 236 in any other suitable manner. Still alternatively, the pull wires 250F, 250S, can be positioned on an exterior of the guidewire lumen 236. Additionally, and/or alternatively, portions of the pull wires 250F, 250S, can be positioned within any other suitable structure of the balloon catheter 218, such as a catheter shaft 234, for example.

In various embodiments, the pull wires 250F, 250S, can have a circular cross-section. In alternative embodiments, the cross-section of the pull wires 250F, 250S, can have any other suitable design. Further, the materials from which the pull wires 250F, 250S, are formed can include a metal or a plastic, such as PTFE-coated stainless steel or a para-aramid synthetic fiber, as non-exclusive examples. Alternatively, the pull wires 250F, 250S, may be formed from any other suitable material or materials.

The design and/or configuration of the steering anchor 252 can vary. In one non-exclusive embodiment, the steering anchor 252 can have a circular or ring-shaped configuration. In alternative embodiments, the steering anchor 252 can include any other suitable configuration. In various embodiments, the steering anchor 252 can be positioned at any location on or within the guidewire lumen 236. In this embodiment, the steering anchor 252 is positioned within the distal region 242 of the guidewire lumen 236.

Additionally, the steering anchor 252 can be coupled, secured or connected to the balloon catheter 218, which may include the guidewire lumen 236. More specifically, in some embodiments, the steering anchor 252 can be coupled, secured or connected to the distal region 242 of the guidewire lumen 236. As certain non-exclusive examples, the steering anchor 252 can be coupled, secured or connected to the interior or the exterior of the guidewire lumen 236. While in the embodiment illustrated in FIG. 2, the steering anchor 252 is shown in phantom at the interior of the guidewire lumen 236, it is understood that in other embodiments the steering anchor 252 can be coupled, secured or connected to the exterior of the guidewire lumen 236. In other words, FIG. 2 is not intended to be limiting in any manner. The steering anchor 252 can be coupled, secured or connected to the guidewire lumen 236 with the use of an adhesive or a thermal bonding technique, as non-exclusive examples. Alternatively, the steering anchor 252 may be coupled, secured or connected to the balloon catheter 218 in any other suitable manner which allows the operator to articulate the guidewire lumen 236 as desired. Additionally, the steering anchor 252 may be made from any suitable material or materials.

In various embodiments, the pull wires 250F, 250S, can be coupled, secured and/or connected to the steering anchor 252. The pull wires 250F, 250S, may be coupled, secured and/or connected to the steering anchor 252 in any suitable manner, i.e., weld or solder joint, adhesive, bonding material, etc.

The steering anchor 252 can be positioned at any location along the length of the balloon catheter 218, including within the interior of the guidewire lumen 236. For example, the steering anchor 252 can be positioned distally (away from) from the handle assembly 220 along a portion of the balloon catheter 218, such as at or near the distal region 242 of the guidewire lumen 236. As illustrated in FIG. 2, the steering anchor 252 can be positioned at the distal region 242 of the guidewire lumen 236, and more specifically near the proximal end 244P of the distal region 242. Alternatively, the steering anchor 252 can be positioned at any location within the distal region 242 of the guidewire lumen 236.

In various embodiments, the steering assembly 226 can allow the operator to articulate the guidewire lumen 236. More specifically, the steering mechanism 248 can be actuated to cause the movement, i.e., push or pull motion, of the pull wires 250F, 250S. In certain embodiments, movement of the pull wires 250F, 250S, can be realized by exerting and/or applying a force, i.e., tension or compression, on the pull wires 250F, 250S. In one embodiment, this movement can function to simultaneously pull or tighten the first pull wire 250F while pushing or loosening the second pull wire 250S, and vice versa. In alternative embodiments, the steering mechanism 248 can be actuated to exert and/or apply force on the pull wires 250F, 250S, via any other suitable manner. Additionally, and/or alternatively, the steering mechanism 248 can be actuated to move the pull wires 250F, 250S, via any suitable manner that allows the operator to articulate the guidewire lumen 236 as desired during the cryoablation procedure.

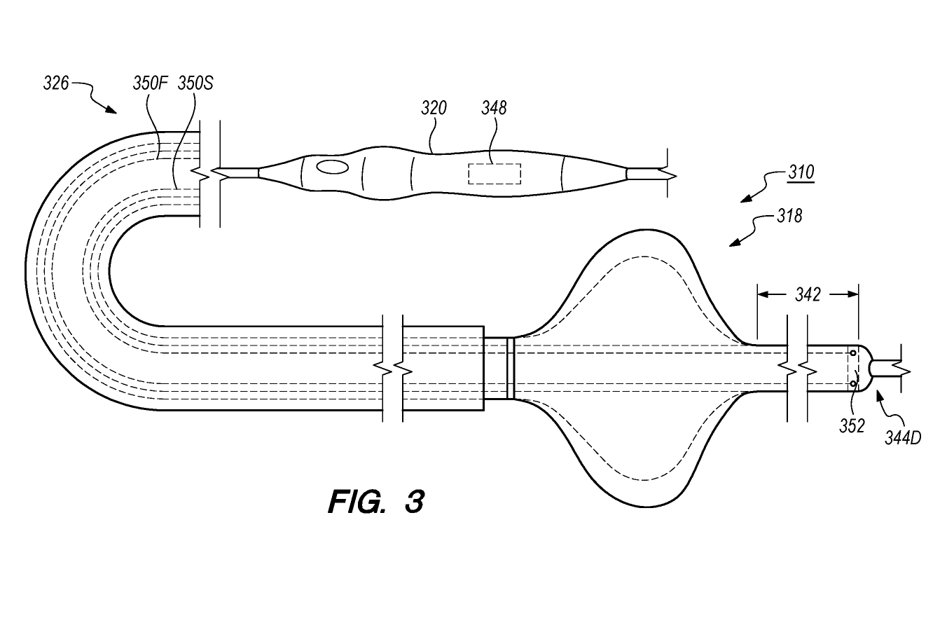

FIG. 3 is a simplified side view of another embodiment of a portion of the catheter system 310, including another embodiment of the steering assembly 326. In the embodiment illustrated in FIG. 3, the catheter system 310 includes the balloon catheter 318, the handle assembly 320 and the steering assembly 326.

In the embodiment illustrated in FIG. 3, the steering assembly 326 includes the steering mechanism 348, the first pull wire 350F, the second pull wire 350S and the steering anchor 352. In this embodiment, the pull wires 350F, 350S, extend between the handle assembly 320 and the distal region 342. More specifically, the pull wires 350F, 350S, extend between the handle assembly 320 and to a location at or near the distal tip 344D of the distal region 342.

Additionally, in FIG. 3, the pull wires 350F, 350S, are coupled, secured and/or connected to the steering anchor 352. The steering anchor 352 is positioned within the distal region 342 at or near the distal tip 344D. Alternatively, the steering anchor 352 can be positioned at any other location within the distal region 342. Additionally, the steering anchor 352 is also coupled, secured or connected to the distal region 342. While the steering anchor 352 is shown in phantom at the interior of the guidewire lumen 336, the steering anchor 352 can be coupled, secured or connected to the interior or the exterior of the guidewire lumen 336. Stated another way, FIG. 3 is not intended to be limiting in any manner.

It is understood that although a number of different embodiments of the steering assembly of the catheter system have been illustrated and described herein, one or more features of any one embodiment can be combined with one or more features of one or more of the other embodiments, provided that such combination satisfies the intent of the present invention.

While a number of exemplary aspects and embodiments of the steering assembly of the catheter system have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A steering assembly for an intravascular catheter system, the intravascular catheter system including an inflatable balloon and a guidewire lumen that extends through the inflatable balloon, the guidewire lumen having a distal region, the steering assembly comprising:
a steering mechanism that is positioned away from the inflatable balloon;
a steering anchor secured to the distal region of the guidewire lumen; and
a first pull wire that is secured to the steering anchor and coupled to the steering mechanism so that actuation of the steering mechanism articulates the distal region of the guidewire lumen.

2. The steering assembly of claim 1 wherein the intravascular catheter system includes a handle assembly, and wherein the steering mechanism is positioned within the handle assembly.

3. The steering assembly of claim 1 wherein the distal region of the guidewire lumen includes a distal tip, and wherein the steering anchor is secured to the distal tip.

4. The steering assembly of claim 1 wherein the steering anchor is secured to an interior of the guidewire lumen.

5. The steering assembly of claim 1 wherein the steering anchor is secured to an exterior of the guidewire lumen.

6. The steering assembly of claim 1 wherein the first pull wire is positioned within an interior of the guidewire lumen.

7. The steering assembly of claim 1 wherein the steering mechanism moves the first pull wire to articulate the distal region of the guidewire lumen.

8. The steering assembly of claim 1 further comprising a second pull wire that is secured to the steering anchor and coupled to the steering mechanism.

9. The steering assembly of claim 8 wherein the distal region of the guidewire lumen includes a distal tip, and wherein the steering anchor is secured to the distal tip.

10. The steering assembly of claim 8 wherein the steering anchor is secured to an interior of the guidewire lumen.

11. The steering assembly of claim 8 wherein the steering anchor is secured to an exterior of the guidewire lumen.

12. The steering assembly of claim 8 wherein the first pull wire and the second pull wire are positioned within an interior of the guidewire lumen.

13. The steering assembly of claim 8 wherein the steering mechanism moves the first pull wire and the second pull wire to articulate the distal region of the guidewire lumen.

14. A steering assembly for an intravascular catheter system, the intravascular catheter system including an inflatable balloon and a guidewire lumen that extends through the inflatable balloon, the guidewire lumen having a distal region, the steering assembly comprising:
a steering mechanism that is positioned away from the inflatable balloon;
a first pull wire that is coupled to the steering mechanism and connected to the distal region of the guidewire lumen; and
a second pull wire that is coupled to the steering mechanism and connected to the distal region of the guidewire lumen;
wherein actuation of the steering mechanism articulates the distal region of the guidewire lumen.

15. The steering assembly of claim 14 wherein the distal region of the guidewire lumen includes a distal tip, and wherein the first pull wire and the second pull wire are connected to the distal tip.

16. The steering assembly of claim 14 wherein the first pull wire and the second pull wire are positioned within an interior of the guidewire lumen.

17. The steering assembly of claim 14 wherein the steering mechanism moves the first pull wire and the second pull wire to articulate the distal region of the guidewire lumen.

18. The steering assembly of claim 14 further comprising a steering anchor positioned within the distal region of the guidewire lumen.

19. The steering assembly of claim 18 wherein the steering anchor is secured to one of an interior and an exterior of the guidewire lumen.

20. A steering assembly for an intravascular catheter system, the intravascular catheter system including a handle assembly, an inflatable balloon and a guidewire lumen that extends through the inflatable balloon, the guidewire lumen having a distal region, the steering assembly comprising:
a steering mechanism that is positioned within the handle assembly;
a steering anchor positioned within the distal region of the guidewire lumen and secured to one of an interior and an exterior of the guidewire lumen;
a first pull wire that is secured to the steering anchor and coupled to the steering mechanism;
a second pull wire that is secured to the steering anchor and coupled to the steering mechanism;
wherein actuation of the steering mechanism articulates the distal region of the guidewire lumen.
